(19) United States
(12) Patent Application Publication
(10) Pub. No.: US 2013/0276792 A1
(43) Pub. Date: Oct. 24, 2013

(54) CERVICAL CAP

(71) Applicant: Joseph W. Moch, Costa Mesa, CA (US)

(72) Inventor: Joseph W. Moch, Costa Mesa, CA (US)

(21) Appl. No.: 13/848,399

(22) Filed: Mar. 21, 2013

Related U.S. Application Data
(60) Provisional application No. 61/614,362, filed on Mar. 22, 2012.

(51) Int. Cl. A61F 6/14

(52) U.S. Cl. CPC A61F 6/146
USPC 128/841; 264/328.1

(57) ABSTRACT

A cervical cap is provided having an annular rim with a first predetermined thickness. An annular collar extends from the rim and has a second predetermined thickness that is greater than the first predetermined thickness. A dome shaped wall extends from the collar and has a third predetermined thickness that is less that the second predetermined thickness. A method of forming a cervical cap is provided that includes the steps of preparing a three part mold, injecting material into the mold, and curing the material in the mold to form the cervical cap.

### CROSS-REFERENCE TO RELATED APPLICATIONS

This application claims priority to U.S. Provisional Patent Application 61/614,362 filed on Mar. 22, 2012, which is hereby incorporated by reference in its entirety.

### TECHNICAL FIELD

The present disclosure relates generally an intra-vaginal barrier device for females, and more particularly to a multi-purpose cervical cap, which fits over the cervix.

Various types of intra-vaginal barrier devices that fit over the female cervix are presently available. These devices include a cervical cap, a diaphragm, a female condom or the like. A barrier device, such as a cervical cap may have several purposes. For example, the cervical cap may serve as a barrier method of contraception to prevent semen from entering the cervical canal. In another example, the cervical cap prevents the transmission of harmful bacteria related to sexually transmitted disease. The cervical cap may also be utilized in applying medicine locally to the cervix or uterus. The cervical cap is retained in place within the vaginal canal by a suction effect that formed a seal between an edge or rim of the cervical cap and the vaginal wall.

Thus, to remove the cervical cap, the suction between the cervical cap and vaginal wall must be broken. Various techniques are available to displace the cervical cap, ranging from prying an instrument such as a finger between the cervical cap and the vaginal wall, to exerting pressure on a handle portion of the cervical cap. Previously, the handle projected from the rim of the cervical cap, since manufacturing constraints limited a more accessible placement of a removal handle.

Thus, there is a need in the art for a cervical cap having a removal handle that can be molded in a single operation. Also, there is a need for a cervical cap that can be comfortably worn for extended periods of time, and accommodates a variety of anatomical sizes and shapes.

### SUMMARY

A cervical cap is provided having an annular rim with a first predetermined thickness. An annular collar extends from the rim and has a second predetermined thickness that is greater than the first predetermined thickness. A dome shaped wall extends from the collar and has a third predetermined thickness that is less that the second predetermined thickness. A method of forming a cervical cap is provided that includes the steps of preparing a three part mold, injecting material into the mold, and curing the material in the mold to form the cervical cap. A removal handle having a first end that is integrally formed with the collar and a second opposed end that is integrally formed with an opposite side of the collar, wherein the removal handle is spaced a predetermined distance from the base.

Accordingly, an advantage of the present disclosure is that a cervical cap is provided that can be used for multiple purposes, including as a contraceptive device, a barrier to prevent a sexually transmitted disease, a drug delivery article, an implantation aid, to assist in regeneration of cervical tissue, a therapeutic device, a sample collection device or the like. Another advantage of the present disclosure is that the cervical cap can be easily removed. A further advantage of the present disclosure is that the cervical cap can be reliably molded in a cost effective manner. Yet a further advantage of the present disclosure is that the cervical cap includes a removal handle that is integrally formed with the cap wall. Still yet a further advantage of the present disclosure is that use of the device is not limited to human applications. Another advantage of the present disclosure is that the cervical cap has a flexible rim to accommodate a variety of anatomical sizes and shapes.

Other features and advantages of the present disclosure will become readily appreciated as the same becomes better understood after reading the following description when considered in conjunction with the accompanying drawings.

### BRIEF DESCRIPTION OF THE DRAWINGS

FIG. 1 is an elevational front view of a cervical cap according to the present disclosure.

FIG. 2 is an elevational side view of the cervical cap of FIG. 1

FIG. 3 is a cross-sectional view of the cervical cap of FIG. 2.

FIG. 4 is an end view of the cervical cap of FIG. 2.

FIG. 5 is a perspective view of the cervical cap of FIG. 1.

FIG. 6 is another perspective view of the cervical cap of FIG. 1.

FIG. 7 is a side view of the cervical cap of FIG. 1.

FIG. 8 is another perspective view of the cervical cap of FIG. 1.

FIG. 9 is another perspective view of the cervical cap of FIG. 1 showing the parting lines for molding purposes.

FIG. 10 is a side view of the cervical cap of FIG. 1 showing the parting lines for molding purposes.

FIG. 11 is an enlarged edge detail view of the cervical cap of FIG. 1.

FIG. 12 is a perspective view of the interior of the cervical cap of FIG. 1.

FIG. 13 is an end view of the interior of the cervical cap of FIG. 1.

FIG. 14 is a flowchart of a method of forming a cervical cap.

FIG. 15 is a flowchart of method of using a cervical cap.

### DETAILED DESCRIPTION

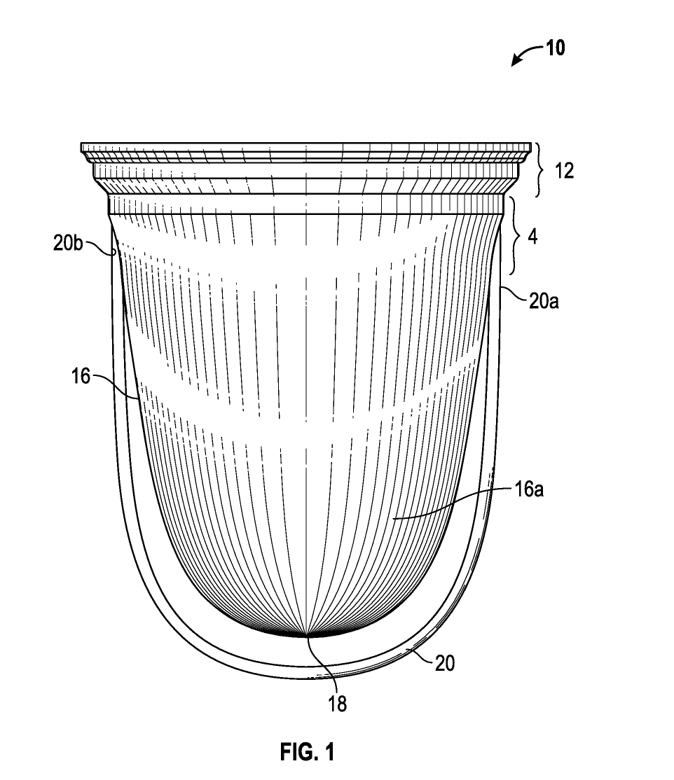

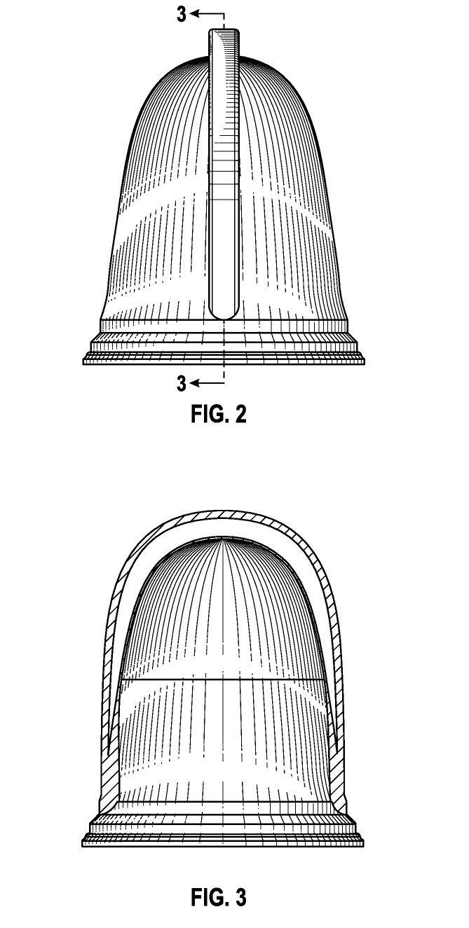

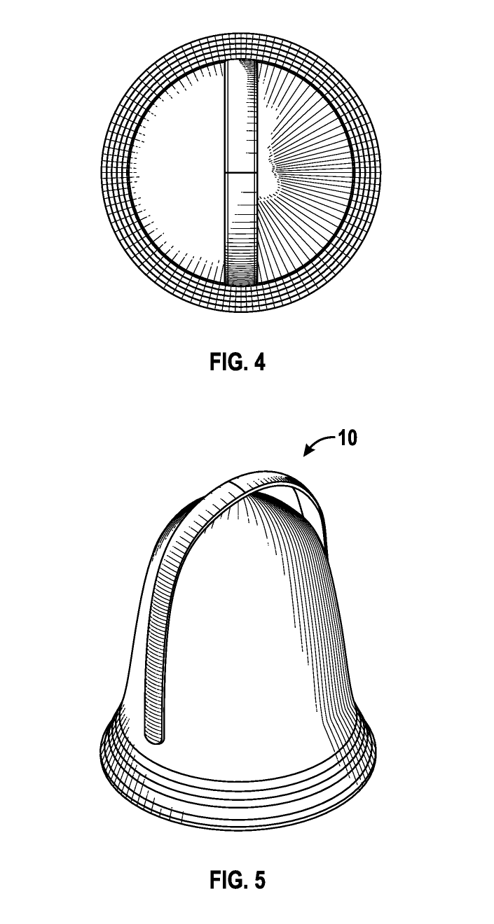

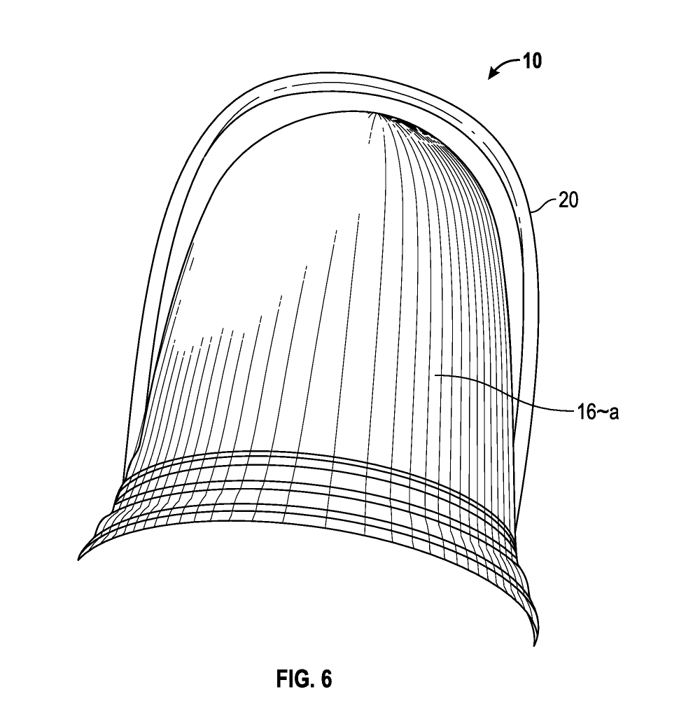

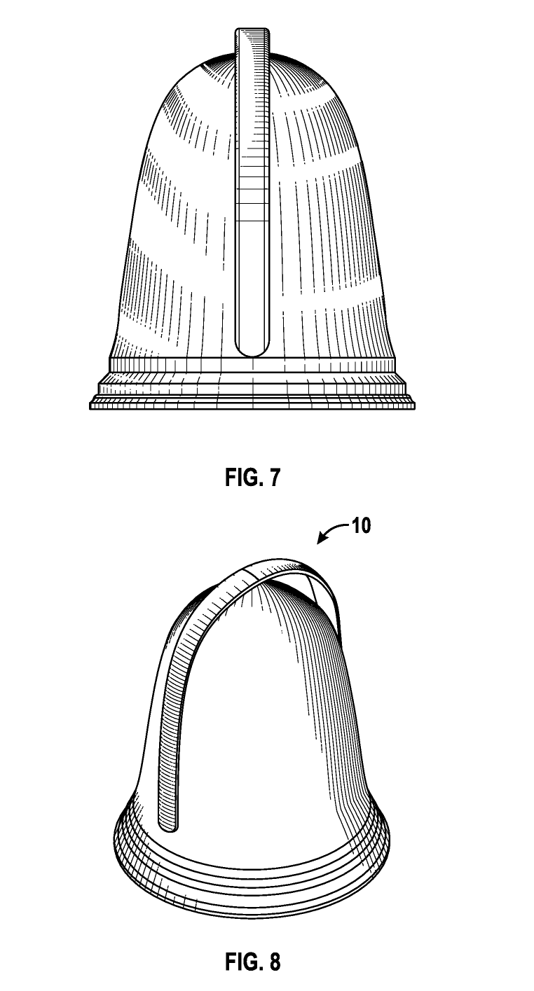

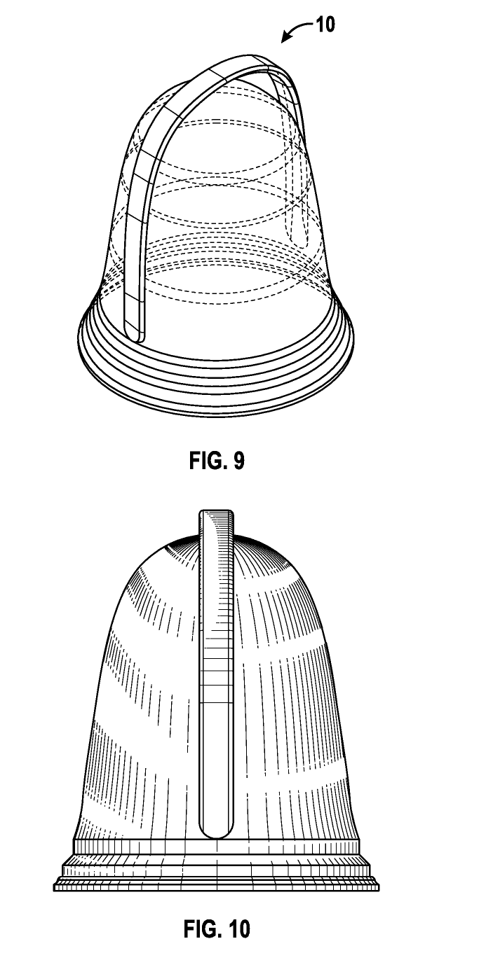

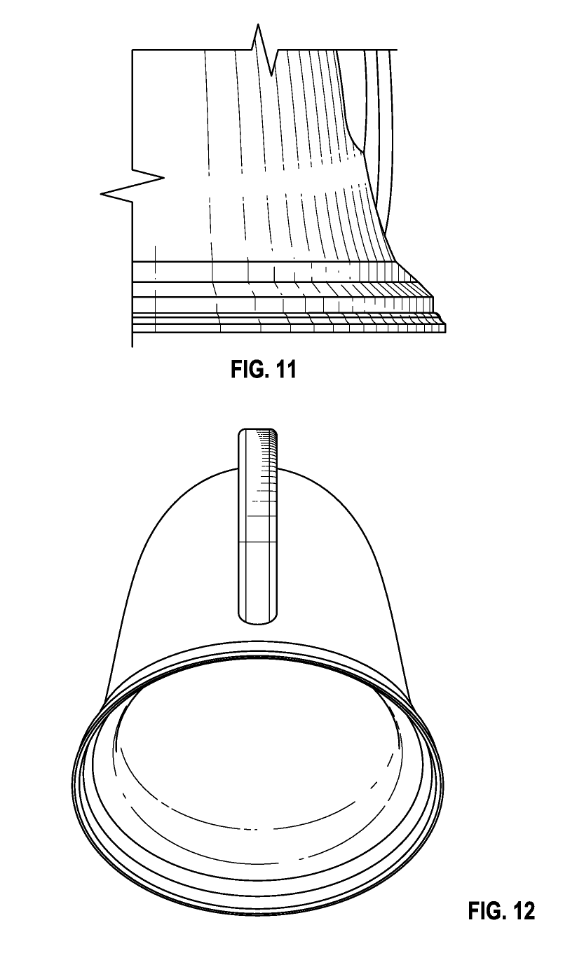

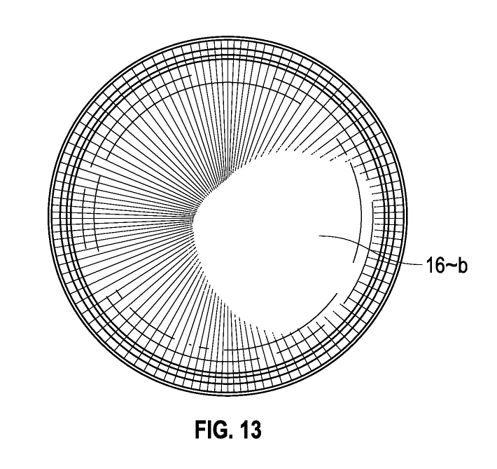

Referring to FIGS. 1-13 a cervical cap 10 is illustrated. The cervical cap 10 of this example has many different uses, from disease prevention to a contraceptive barrier device, to a clinical sample collection device. Further, the cervical cap 10 can be placed in any type of female, including humans and other animals.

The cervical cap 10 includes an annular rim 12 that forms a flange for retention of the cervical cap 10 around the cervix. The cervical cap 10 also includes a dome or cup shaped wall that is continuous with the rim 12. The dome wall 16 and rim each have an external surface 16a and an interior surface 16b. The interior surface of the dome wall is accessible via a central opening formed in a center of the rim. The rim 12 has at least one integrally formed step that is progressively reduced in diameter from an outermost edge of the rim. In this example, there are five steps. The thin, stepped rim flange 12 rises upward from the collar 14 diagonally at a predetermined angle, such as 65 degree. The rim 12 can have another profile, such as an accordion pattern or the like. The rim has a predetermined height, such as 4 mm.

A portion of the dome wall 16 that is immediately adjacent the rim is thicker in diameter that the rest of the dome wall and forms an annular collar 14. The collar 14 may have a length of about 10 mm. The remaining portion of the dome wall 16 is thinner in diameter than the collar portion in a manner to be described. The end of the dome wall forms a base for the cervical cap, as shown at 18.

The cervical cap 10 also includes an integrally formed removal handle 20. The removal handle 20 is generally planar and includes a first end and a second end. The first end 20a of the removal handle 20 is integrally secured to the outer surface of the dome wall just below the rim, in the vicinity of the collar. The second end 20b of the removal handle is integrally secured to the outer surface of the dome wall on an opposite to the first end. Thus, a first portion of the removal handle is integrally formed with the dome wall and as shown at 20a, and a second portion of the removal handle is spaced apart from the dome wall as shown at 20b. The removal loop 20 is spaced from the base of the dome wall to accommodate grasping. For example, the removal handle may be approximately 4 mm in width, and approximately 1 mm in thickness.

The circumference of the rim 12 can vary depending on the width of the cervix to which it is secured. Similarly, the length of the dome wall 16 varies depending on the anatomical characteristics of the cervix. For example, the diameter of the interior of the cervical cap is about 28 mm at the collar 14, and about 42 mm in height from an edge of the rim to the base 18. Advantageously, the rim 12 is flexible and can accommodate multiple cervical sizes and shapes.

The thickness of the dome wall 16 varies along the length of the dome wall. For example, the thickness of the rim is about 0.25 mm. In the area of the collar and integrally formed handle, the thickness is approximately 3 mm. As the handle 20 diverges from the dome wall, the thickness of the dome wall is reduced to approximately 0.25 mm. The thin, stepped rim flange 12 gradually tapers to a thickness of less than 0.25 mm at the outer edge of the opening 10.

The diameter of the cup 16 may be narrowest at the base, and gradually widens toward the opening formed by the rim, such as approximately 20 mm near the base, approximately 28 mm inside the thick collar 14, and approximately 39 mm at the outer edge of the rim flange 12.

The cervical cap 10 can be made from various flexible medical grade materials. For example, medical grade silicone, such as latex, plastic and the like may be utilized. Depending on the use of the cervical cap, it may be desirable that the material be impermeable. I.e. the material could be impermeable to sperm and sexually transmitted viruses or bacteria and not impermeable to drugs or hormones. Alternatively, the wall of the cup portion may be permeable to a spermicidal preparation contained within the cup portion, but impermeable to sperm. In this manner, the spermicide may expand within the vagina and around the cervix. In still another example, based on the principle of occlusion, the cervical cap 10 would be put in place over the cervix, and the cup material would be impenetrable to STI pathogens and, resultantly, the device would act as a physical barrier to offer protection from certain sexually transmitted infections, such as HIV/AIDS, HPV, Chlamydia, Gonorrhea, and Syphilis. In still another example, based on the inclusion of a microbicide or other chemical medicament, the cap would act as a physical and chemical barrier to offer protection from certain sexually transmitted infections, such as HIV/AIDS, HPV, Chlamydia, Gonorrhea and Syphilis.

In a further example, the predetermined material placed in the cup portion of the cap could be a drug or hormone-loaded delivery system. Certain cup construction materials may be permeable in a way that they can be loaded with drugs and/or hormones. Accordingly, the cervical cap would become a mechanism for the intra-vaginal, controlled release of a range of drugs and/or hormones, where local or trans-dermal/trans-mucosal delivery is preferred. In still another example of a conception aid, the predetermined material is live sperm be placed over the cervix to directly deliver that sperm to the uterus, or opening of the cervix, to facilitate conception. In yet another example, the cervical cap 10 could be placed on the cervix to protect and enable regeneration of the cervical tissue, with, or without added material or medication. Similarly, as a protective device, the cervical cap could be used therapeutically to protect the cervix, such as prior to or after a fertility treatment.

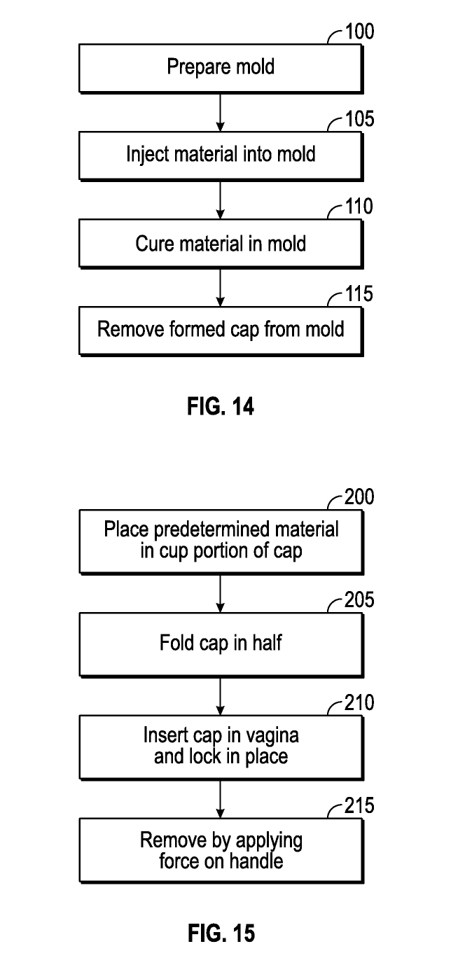

Referring to FIG. 14, a method of molding a cervical cap 10 is provided. The methodology begins in block 100 with the step of preparing a precision mold. For example, a mold is prepared using a high precision CNC machine such as a 3D printer/rapid prototyping machine is utilized. The mold will have 3 pieces that splits across two distinct parting lines as shown in FIG. 8.

The methodology advances to block 105 and a material is injected into the mold. An example of a material is a medical grade flexible material.

The methodology advances to block 110 and the material is cured for a predetermined period of time and at a predetermined temperature and pressure.

The methodology advances to block 115 and the molded cervical cap is removed from the mold.

The methodology may includes other steps that are conventionally known in the art of molding, such as cooling, coating or the like.

Referring to FIG. 15, a method a using the cervical cap as a contraceptive device is illustrated. The method begins at 200 with the step of optionally placing a predetermined material in the cup portion of the cap. At step 205, the cervical cap is folded in half lengthwise and inserted, open-end first, into the vagina, and guided up the vaginal canal. At step 210, the cervical cap is placed over the cervix at the far inner end of the vagina so that the cup portion completely covers the cervix, and the thin, stepped rim flange encircles the cervix and is nestled up against the cul de sac of the vagina, at the fornix. The thin, pliable material of the cup effectively lies against, or “marries” up against the surface of the cervix via surface adhesion to retain the device against the cervix. The rim forms a seal against the fornix and/or the vaginal wall at the cul de sac of the back of the vagina, by way of adjusting to the depth of the space, between the cervix and the vaginal wall, terminating as the fornix. The rim automatically adjusts as necessary to the varied depth at the fornix, to flexibly reach to, and seal against it. The seal that is formed at the fornix provides an added barrier to sperm, blocking possible travel of the sperm into the inner side of the device, and ultimately to the cervix and uterus. Further, the seal “locks” the device in place until that seal is interrupted by exerting an opposite force on the removal loop. The device could stay in place for an extended period of time, as needed, possibly up to several days. Once removed, the cervical cap may be cleaned and reused. It should be appreciated that the cup portion of the cap may be filled with a material have a specific purpose prior to insertion. For example, a spermicidal may be placed inside the cup portion of the cervical cap. The methodology advances to step 215 and a force is applied to the handle in order to remove the cervical cap 10.

The present disclosure has been described in an illustrative manner. It is to be understood that the terminology which has been used is intended to be in the nature of words of description rather than of limitation. Many modifications and variations of the present example are possible in light of the above teachings. Therefore, within the scope of the appended claims, the present disclosure may be practices other than as specifically described.

What is claimed as new and desired to be protected by Letters Patent of the United States is:

1. A cervical cap comprising:
an annular rim having a first predetermined thickness;
an annular collar extending from the rim and having a second predetermined thickness that is greater than the first predetermined thickness;
a dome shaped wall extending from the collar and having a third predetermined thickness that is less that the second predetermined thickness;
a removal handle having a first end that is integrally formed with the collar and a second opposed end that is integrally formed with an opposite side of the collar, wherein the removal handle is spaced a predetermined distance from the base.

2. A method for forming a cervical cap, said method comprising the steps of:
forming a three part mold;
injecting material into the mold; and
curing the material to form the cervical cap having an annular rim with a first predetermined thickness, an annular collar extending from the rim and having a second predetermined thickness that is greater than the first predetermined thickness, a dome shaped wall extending from the collar and having a third predetermined thickness that is less that the second predetermined thickness, and a removal handle having a first end that is integrally formed with the collar and a second opposed end that is integrally formed with an opposite side of the collar, wherein the removal handle is spaced a predetermined distance from the base.
